(19) United States
(12) Patent Application Publication
(10) Pub. No.: US 2019/0231578 A1
(43) Pub. Date: Aug. 1, 2019

(54) CUSTOMIZED SPLIT INSOLE FOR DIABETIC PATIENTS

(71) Applicant: Rangasamy Naidu Educational Trust, Peelamedu (IN)

(72) Inventors: Rajesh Ranganathan, Coimbatore (IN); Sivakumar Ganesan, Coimbatore (IN)

(21) Appl. No.: 15/882,442

(22) Filed: Jan. 29, 2018

(51) Int. Cl. A61F 5/14; A43B 17/00

(52) U.S. Cl. CPC A61F 5/14; G06F 17/50; A43B 17/00

(57) ABSTRACT

An inventive custom made split insole for use by diabetic patients, split into zones for normal and flat foot persons and five zones for high arch foot persons where these splits have projections which are hexagonal or oval in shape and have specific dimensions and specific spacing but are of uniform height and elasticity.

### FIELD OF INVENTION

The present invention pertains to production of customized split insole for use by diabetic patients so as to prevent or reduce the chance of formation of foot ulcers and thereby gangrene and amputation.

### BACKGROUND OF THE INVENTION

Lower most part of human body is the foot, which provides balance, supports in mobility and makes human to do numerous activities like walking, running, jumping, climbing and other activities (Healthline, 2015). The complex structure of foot comprises of 26 bones and 33 joints, ligaments, muscle and more than hundred tendons working together to allow the foot to adjust to walk on the uneven surfaces either it is even or uneven, and acts as a shock absorber (Watt, 2009). The interaction between the foot and ground are very important in designing the footwear and orthotic devices (Albert et al., 2011). The ill fit shoe or orthotic device design causes high pressure in the plantar surface of the foot, which interfaces foot and footwear or foot and ground surface. Peak pressure in the contact area leads to foot deformities, ulcers, corns, calluses and bunions (Dunn et al., 2004).

According to International Diabetes Federation (IDF, 2013), 387 million people have diabetes in 2013 and this is expected to rise to 592 million by 2035. Seventy seven percentage of the diabetic people live in low and middle-income countries, every seven seconds a person dies from diabetes and it caused 5.1 million deaths in 2013. Diabetes impacts the humans primarily on three organs, namely kidneys, retina and foot. Foot problems in a person with diabetes can have severe consequences. A study conducted on diabetic foot amputation by Amit kumar et al. (2010), revealed that out of 114 patients 48 had undergone foot amputation. (Hadi et al. 2012), concentrating on foot, revealed that foot forms a primary contact with ground surface and there is a great care to be taken in the plantar region of the foot. Ill-fitting footwear causes injury and deformity and specific care is to be taken while selecting the shoe types and shoe fitting (Watt, 2009).

The primary reason for amputation was identified to be because of lack in preventive measures to reduce the burden of ulceration and amputation in diabetic foot. Discussing about the foot ulceration, it was found to be present in feet of the patients. Stokes et al., (1975) used a segmental force platform to study 37 feet of 22 diabetic patients. High loads were found at the sites of ulcers. Patients with high loads under the feet were also heavier in weight than those with lower loads. Toe loads in patients with ulcers were found to be reduced. A shift of maximum loads to the lateral foot in neuropathic patients was also reported.

With loads that are exerted and pressure that emerges underneath the feet playing major role leading to amputation of the foot/portion of the foot or limb itself. Boulton et al., (1983) employed the optical pedobaragraph for research purposes to examine the relationship between high foot pressures and ulceration. In their study, diabetic patients with and without neuropathy and individuals without diabetes were examined to evaluate the relationships among foot pressures, neuropathy, and foot ulceration. Their results demonstrated that a significantly larger number of patients with diabetic neuropathy had abnormally high foot pressures compared with controls. Furthermore, patients with a previous history of foot ulceration had high pressures at ulcerative sites. As ulceration occurred at sites of high plantar foot pressures, therefore, foot pressure reduction should lead to a reduced incidence of foot ulceration in neuropathic diabetic patients.

Duckworth et al. (1985) working on plantar pressure measurement identified that foot ulcer occur at high pressure regions, and inorder to detect them they suggested static and dynamic pressure measurement devices to measure plantar surface pressure points. In order to measure plantar pressure distribution, Wolfe et al., (1991) suggested, ‘Plantar Pressure Measuring Device’ which is clinically used to scrutinize the asymmetry of plantar pressure distribution in young adults with ankle fractures. They also suggested the use of the same device to measure plantar pressure in diabetic patients with Charcot neuroarthropathy. They used these measures to correct the gait pattern in accident victims, to analyse orthotic problems only and not for any other purpose.

Every person requires an individual assessment of the foot and a customized insole to avoid foot related problem (Zequera and Solomonidis, 2010). Material and shape of the customized insole plays key role in redistribution of plantar peak pressure (Cheung and Zhang, 2007, Tsung et al., 2004).

The custom made insole with a medial arch support may transfer load from the forefoot to the midfoot compared with a flat insole (Bus et al., 2004). The significance of providing arch support is that, it will help in offloading the peak pressures across different anatomical zones. Brown et al. (1996) also found increased midfoot peak pressures in 10 healthy subjects wearing custom made insole or arch supports.

Many podiatrists follow traditional plaster cast mould manufacturing method to fabricate the orthotic device, while cost are high in this method (Crabtree et al., 2009). The design and development of customized footwear has been in practice for the past three decades. Latest development in the field of CAD/CAM and scanner has accelerated the production of orthotic devices (Parreno, 2007). Crabtree et al. (2009) investigating on manufacturing methodology for personalized sports insole; suggested cryogenic machining method as a novel manufacturing method for manufacturing a personalized sport insole. In this method CNC machining is used to produce CAD model from the scanning and assessment methods enables rapid manufacturing of personalized products.

Ability to incorporate the novelty in the design of orthotic device is limited because of the manufacturing methods (Telfer et al., 2012). Recently, we had performed a comparative study of diabetic and non-diabetic population, data were analyzed with respect to different foot types. Plantar pressure values are same for normal and high arch foot for both population. Flat foot diabetic population had a high pressure in the metatarsal region when compared to non-diabetic population. As remedy to overcome the load distribution, the designer has to achieve an optimal design for an individual, with each individual is unique; different in structure, size, movement and gait pattern. An effort was made to aid the design and development of customized split insole using manual masking/zone splitting of the foot plantar region.

One of the very recent surveys conducted by us over a population size of 261 people, 77% is found to diabetic between the ages of 35 to 65. Focus was towards analysis of foot problems of the diabetic patients. Foot problems in a person with diabetes can have severe consequences. Though, recent advances in the management of these problems have increased our abilities to save the lower limb, the best management lacks in taking preventive measure.

This invention is projected towards analyzing diabetic patients about their levels of complication in their lower limb such as foot ulcer, neuropathy and gangrene. Where, foot ulcer is high in the areas of pressure point in the plantar surface leading to gangrene and amputation of lower limb. The pressure point in the plantar surface in the body is dependent on age, weight, lifestyle and further it varies from patient to patient. As the plantar surface differs from person to person and being not the same foot sole can be used for all. This is because of varying pressure point and individual gait pattern.

The gait pattern of a person has a lot to do with the stress and pressure exerted on the foot. Therefore, the gait pattern directly affects the plantar surface of the foot. In normal person, from childhood to middle age the gait pattern does not have any significant harmful effect on the plantar surface of the foot. However, ageing may cause certain orthotic problems which may directly or indirectly affect the plantar surface of the foot. The build-up of pressure on the plantar foot surface has a direct bearing a plantar foot ulcers, especially in diabetic patients. Foot Ulcers occurs at sites of the high pressure on the plantar surface of the foot.

The human foot consists of three parts namely the hind foot, mid foot and the fore foot. The hind foot consists of two bones one on top of the other. The mid foot consists of five bones packed close together, while the fore foot consists of five metatarsals each with phalanges (toes).

The superior surface of the hind foot forms the ankle joint which articulates with the tibia and fibula in the medial and lateral position respectively.

During walking, the entire body weight is mostly borne by one leg at a time. The fibula bone transmits weight to the talus and to the rest of the foot. During walking, when the body first touches the ground, the calcaneus (Head bone) takes the entire weight. But, however, still some body weight is shared by the other foot as well. Once the heal is firmly on the ground, the other foot leads the ground. The fore foot touches the ground, but usually, the lateral border of the foot takes on the weight first transmitting it through the cuboid bone and the base of the fifth metatarsal. Immediately, thereafter, the whole foot is on the ground. Thus, the body weight is transmitted from the calcaneus or head bone to the cuboid bone to the base of the fifth metatarsal and then the heads of all the five metatarsals. Then, when the other foot swings forward, the heal begins to leave the ground and the whole weight is shifted to the forefoot. Thereafter by a strong contraction of the toes, the body is pushed forward to transfer its weight on the other foot which is now in a stable position to receive the transmitted weight. This is one walking cycle.

Therefore it can be said that in one walking cycle, when the foot comes down, it rests back of the heal, then on the lateral side of the metatarsal and finally on the heads of the metatarsal to push off for the next step.

Similarly, when a person is standing, both foot are on the ground where the foot is like an arch sparing the mid foot from weight bearing.

It is therefore fascinating to analyse how instinct the mechanism of walking or standing or running is. The foot therefore displays several motions like acceleration, deceleration, sudden stop, jumping, twisting, turning, kicking, squatting and so on. During all these motions, the pressure exerted on the plantar foot varies.

In diabetic patients, there are combination of various adverse factors which affect the strength and health of the plantar foot. The end result of these various risk factors are neuropathy and tissue damage.

So unlike normal patients, the foot of a diabetic patient is not prepared to handle the pressure build-up on various areas of the plantar foot during various motions in view of its un-healthy state caused due to the said risk factors. Therefore, build-up of plantar pressure at particular areas of the plantar foot will aggravate the neuropathy or tissue damage thus leading to ulcer formation. One way to avoid or reduce the damages of such ulcer formation is by distributing the pressure build-up on the plantar foot to other non-pressure build-up areas by suitably off-loading the pressure

That apart, During leg movement, there is a interlink articulation of one or more of these bones which exerts pressure at various levels on the plantar surface of the foot. Therefore, the gait pattern of a person directly affects the pressure exerted on various areas of the plantar foot surface through the foot bone leading from the leg bone. It varies from person to person depending on his gait pattern and is not uniform for all. The pressure exerted on the various areas of the plantar surface of foot is called plantar pressure. This can be measured by a sensory device and processed by linking it to a computer.

Diabetic patients have a lot of foot related problems. The simplest formation which can lead to greatest complication and loss of limb is the formation of foot ulcers. It is known that have a reduced healing rate when diabetic patients compared to normal healthy persons. So the formation of a foot ulcer tends to progresses ultimately leading to gangrene and amputation of the phalanges or any other part of the foot or the leg itself in extreme cases.

Irrespective of the cause of formation of foot ulcer in diabetic patients, the exertion of pressure on the various areas of the plantar surface of the foot directly affects ulcer formation. This pressure exertion on the various areas of the plantar surface of foot is based on the gait pattern of the person.

When measuring the plantar pressure of a person, it can be seen that there are many pressure build-up areas on his foot. In some of these areas, the pressure build-up is very high. These areas are more prone to foot ulcer formation. By off-loading these pressure in the pressure build-up areas in particular in the peak pressure build-up areas, ulcer formation can be eliminated if not reduced. Off-loading of the pressure build-up is done by dissipating the build-up pressure to the surrounding areas of the plantar surface so that one particular point is not exposed to the pressure build-up which is the cause of ulcer formation.

Further, in diabetic patients, in view of neuropathy or internal tissue damage due to lack of blood supply or deficit blood supply, capillaries get clogged, leading to pressure build-up which aggregates the chances of tissue injury and ulcer formation. So all the more it is necessary to off-load the pressure build-up on the plantar foot surface of diabetic persons to avoid foot ulcers.

More than 15% of the ulcer results in amputation of foot or limb. The treatment of plantar ulcers is also very complex. So the prevention by off-loading of the plantar pressure surface by using customised insoles is a very effective remedy. The inventive customised split insole addresses this issue in a technically better and high cost effective manner as described herein.

Therefore peak pressure in plantar foot surface of diabetic persons is a common route cause for diabetic foot ulcer, leading to gangrene and amputation. To overcome this problem, many insoles for diabetic patients have been designed the world over. However, these insoles are subject to the following limitations;
a. Are not made to individual requirement.
b. The entire customised insole is of single piece.
c. The elasticity and Shore Hardness Value is uniform throughout the entire insole and not split zone wise.
d. Though there is cushioning effect there is no offloading of pressure build up.
e. The problem faced by the diabetic patients and Doctors is that the present insoles are not very effective in distributing off-loading the plantar pressure build-up because their elasticity, and thickness and density is uniform throughout the insole. Whereas, under the inventive split insole, the elasticity, density and thickness of the insole material is varied zone wise so as to very effectively off-load the pressure build-up than in conventional insole.
f. Some of the customised insoles are jelly type to provide cushioning effect. But, the softness of the jelly is uniform throughout. This has two disadvantages namely that it reduces the grip during dynamic activity and secondly, it does not effectively offload the pressure build-up in the pressure points. Therefore being counter protective to the very purpose to which it is made.
   In the present invention, the material hardness varies between the pressure build-up points and the other areas so as to ensure effective off-loading, of the build-in pressure which is also achieved by effectively increasing the plantar contact area of the foot with the floor.
g. The customised single insoles do not consider the arch support in the foot. Because of which, the pressure distribution to the mid foot is not addressed. The present invention addresses this aspect by specifically considering the arch data and the type of foot in calculating the thickness and Shore Hardness Value of the material to be used for the arch.
h. The loss of material is negligible, therefore reducing the material and processing costs. Therefore, the present invention address these limitations and short comings and ensures effective off-loading of pressure built-up in the pressure areas by varying thickness and Shore Hardness Value of the material zone wise for peak pressure areas in a zone wise.
i. Replacement is high as entire insole has to be replaced, whereas here only the worn-out part has to be replaced.

Further, none of the existing insoles were split insoles focused towards effectively offloading the peak pressure zone wise.

### SUMMARY OF THE INVENTION

The inventive product is a foot insole for use by diabetic patients. It is custom made to suit individual requirements. Each customized insole has five or six splits, five for persons with high arch and six for persons with normal or flat foot.

These splits are of specific thickness and have hexagonal or oval projections of specific dimension, height, elasticity and are placed on the first split.

The elasticity of the split insole is fixed zone wise based on the pressure build-up in that plantar area of the foot.

For high arch persons, two mounts are also provided on the fourth zone and for normal and flat foot persons in the fifth zone.

### BRIEF DESCRIPTION OF THE DRAWINGS

FIG. 1 is types of foot.

FIG. 2 is a split insole for normal foot and flat foot.

FIG. 3 is a split insole split into six different zones.

FIG. 4 is a split insole for high arch foot.

FIG. 5 is a pressure distribution on bare foot.

FIG. 6 is pressure distribution using custom insole.

FIG. 7 is pressure distribution of bare foot and custom split foot insole.

### DESCRIPTION AND WORKING

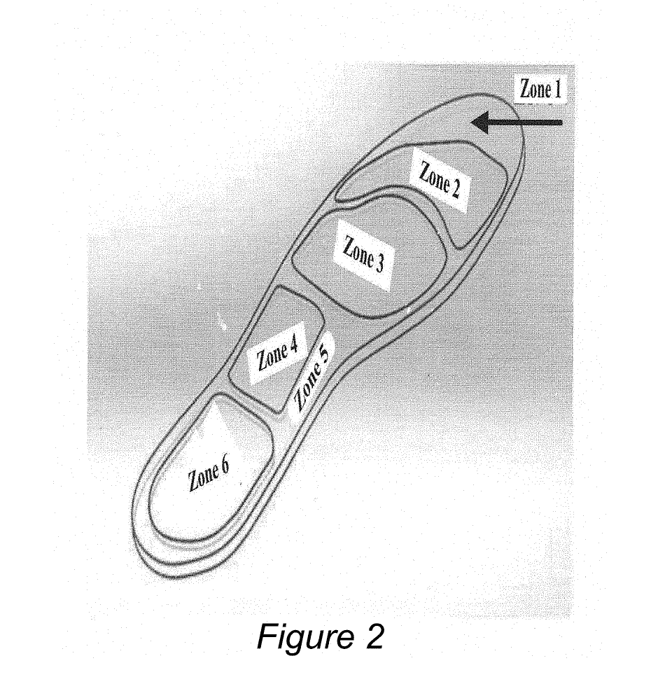

The invention is a customized split insole for use by diabetic patients as in FIG. 2. The foot insole is made from VeroClear RGD810, TangoPlus FLX930 which is a resin. The specialty of this resin is that the shore hardness values can be modified there by changing the elasticity.

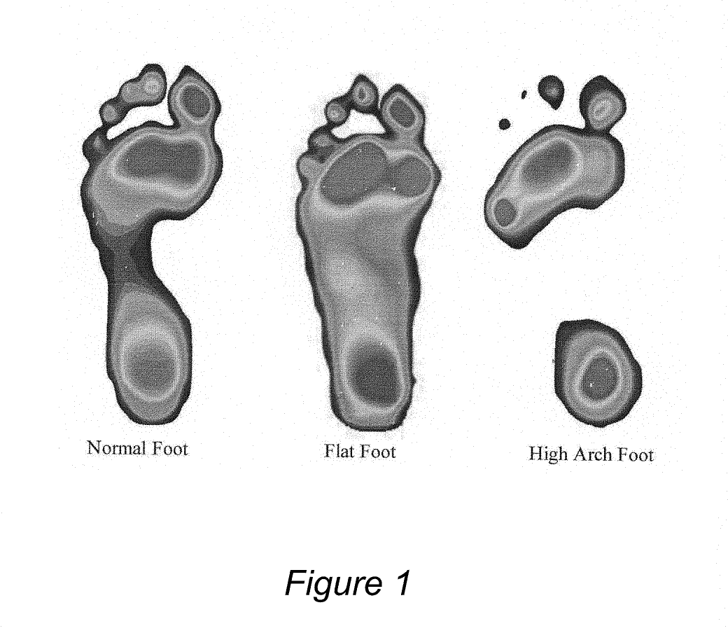

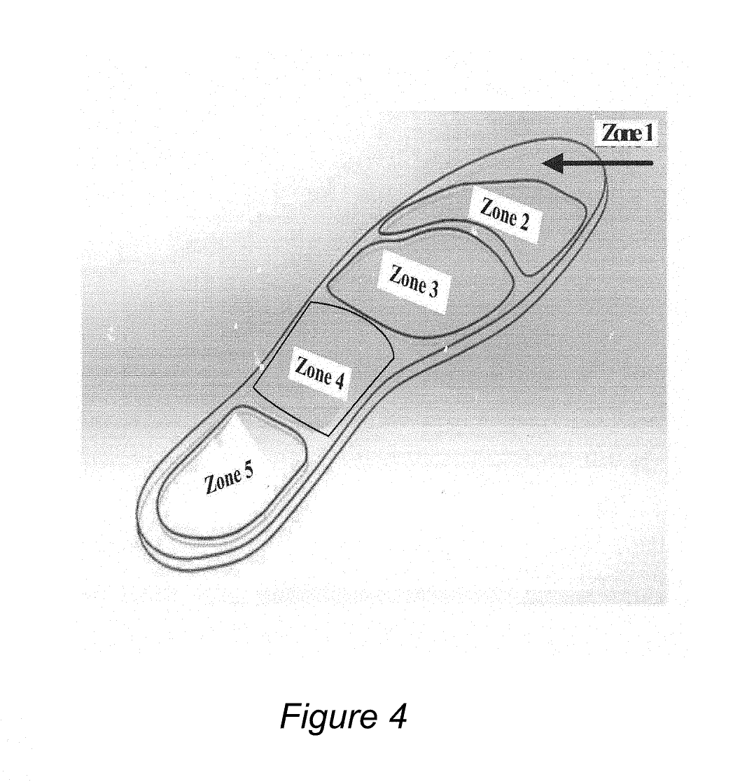

The human foot is classified into three types, namely, normal, flat and high arch foot depending on the arch in the middle inner side of the foot. This is depicted in FIG. 1. The split insole is separately designed in the form of a human foot, for all the said three types of human foot as in FIGS. 2 and 4. Further, the split insole is custom made for each person, so its size varies depending upon the foot size of the person who is to use it.

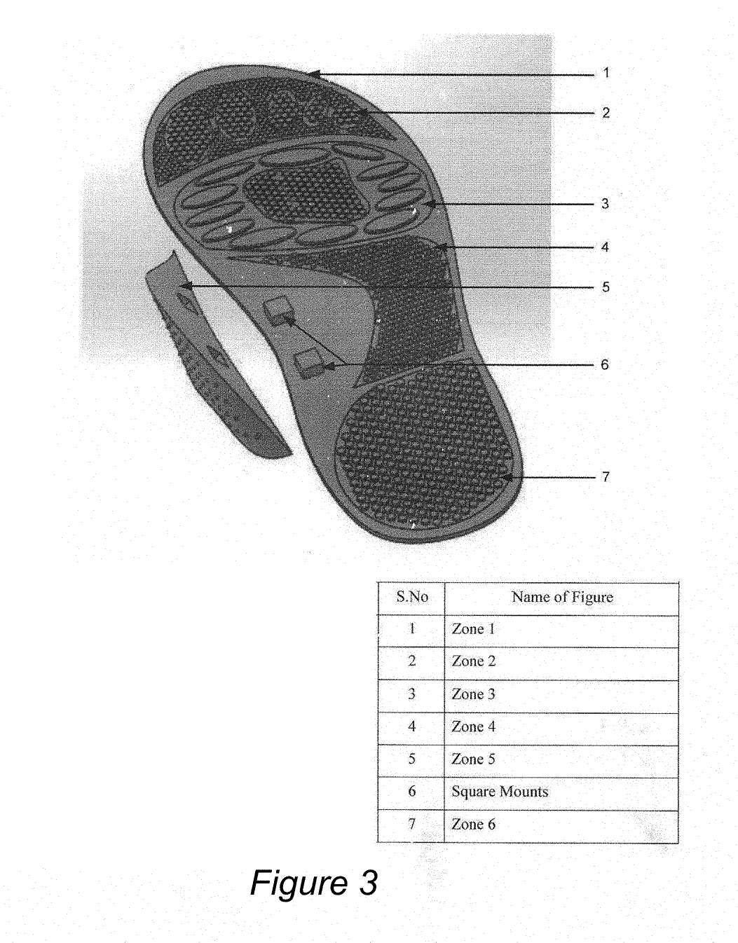

For a normal foot, the split insole is split into six different zones as in FIG. 3. Except the first zone, all the other zones have projections and varying elasticity levels. The shape of each projection is hexagonal and its diameter varies zone wise. However the height of the projections is 1 mm uniform in all the zones. Distance between the projections also varies zone wise. The fifth zone, is arched to fit in to the natural arch of the foot, there are two square mounts molded in zone one, as in FIG. 3.6, into which is fitted the fifth split. Each zone, experiences varied levels of both static and dymanic loads.

The first zone is the bottom zone of the foot insole which acts as a support structure to hold all the other five zones on its top surface, as in FIG. 3.1. This is of uniform thickness and elasticity.

The second zone is located at the head of the split insole, in the phalanges region of the foot, as in FIG. 3.2. The projections are so placed that they are below the person's toes. The projections are made of hexagonal pattern with distance between the hexagons perforation spacing 3 mm and angle 60° and their diameter in this zone is 2.5 mm. The thickness of the insole in this zone is 4 mm. The elasticity, which is the flexibility of the sole measured as the ‘shore hardness value’ varies according to the requirement for this zone. Further, in this zone, depending on the persons pressure build up area, the elasticity is more if the pressure acting is more and vice versa. In this zone also primarily there is pressure distribution.

The third zone is located just below the second zone, in the metatarsal region of the foot, as in FIG. 3.3. The projections are located only in the middle of this zone. Around the projections there are horizontally placed oval projections of varying dimensions. This size and placement of these oval projections depends upon the peak pressure acting on the person's foot. In this zone also primarily there is pressure distribution.

The fourth zone is located just below the third with the fifth zone on its inner side, as in FIG. 3.4. The entire zone is filled with projection till the border of this zone. Height and diameter are even for all the projections, but vary from person to person.

The fifth zone is located on the inner curve of the foot, adjacent to the fourth zone, as in FIG. 3.5. It is curved according to the arch profile of the person's foot. Elasticity is same through out this zone. Projections are given for enhanced gripping and ventilation. The split in this zone has two square sockets in its bottom side, which are so made to fit into the square mounts in the first zone, as in FIG. 3.6.

The sixth zone is at the bottom of the foot insole in the heel region of the foot, and below the fourth zone, as in FIG. 3.7. This zone receives much more pressure from the person, than the other zones. The projections are evenly spaced and of diameter of hexagon is 3 mm, pattern layout—perforation spacing 4.5 mm and angle 60°. Elasticity is high, because the pressure applied in this zone is much higher when compared to the other zones.

For a flat foot person, the split insole is split into six zones similar to the normal foot split insole as in FIG. 2. But here the height of the inner arch is comparatively lower than that in the normal arch, rest of its description is the same as that for a normal insole.

Similarly, for a high arch foot person, the split zone is split into five zones as in FIG. 4. Here the third and fifth zones of a split insole of a normal person are combined into one. Rest of the description and functionality is similar to the normal arch person.

Working:

The custom made split insole is inserted into the foot wear of the person or patient who is to use the same. He can start moving by wearing the footwear inserted with the custom made split insole. The split insole with the projections and elasticity offloads the pressure build up in certain areas to the other parts of the foot, avoiding concentrating of pressure at particular points of the foot which in due course lead to foot ulceration and in several cases to gangrene and amputation in the diabetic patients.

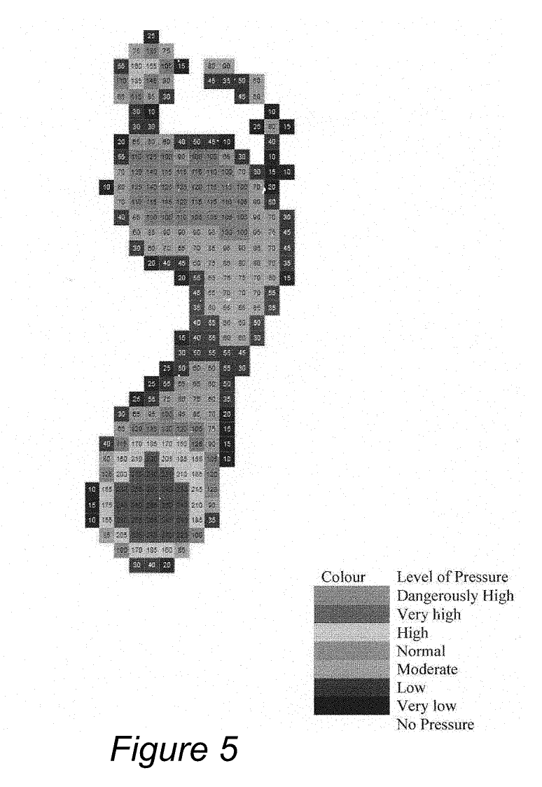

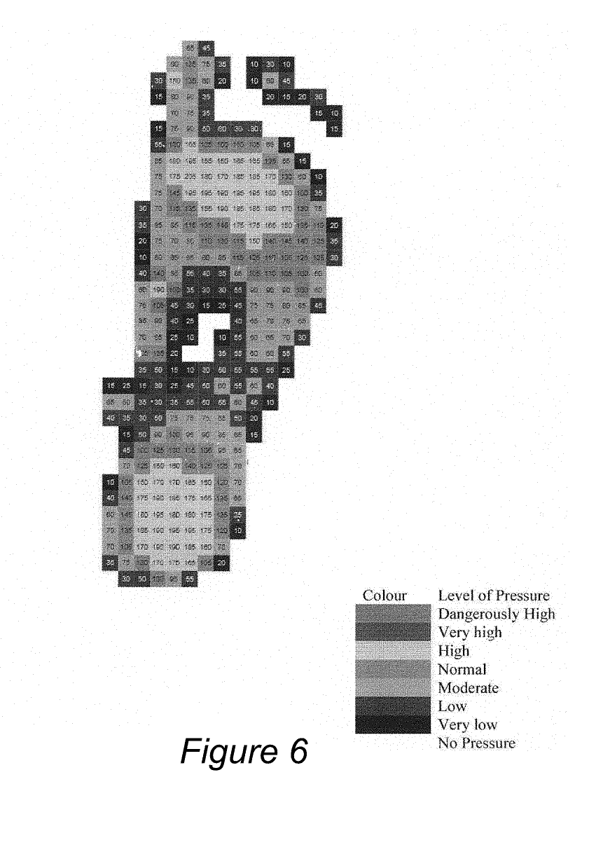

The distribution of peak pressure to the other parts of the foot is measured using the ‘Plantar Pressure Measuring Device’, which is a machine with a sensor pad used for pressure data collection for both static and dynamic situations. The peak pressure of the bare foot as measured by the ‘Plantar Pressure Measuring Device’ is as shown in FIG. 5. The peak pressure distributed to the other parts of the foot when using the custom made split insole is as shown in FIG. 6.

### Example

The distribution of peak pressure loads under bare foot and using the custom split foot insole is as illustrated in the following Table 1;

TABLE 1
Pressure distribution with bare foot and custom split foot insole

| | Bare Foot | | | | | Custom Insole | | | | |
|---|---|---|---|---|---|---|---|---|---|---|
| Zone | 2 | 3 | 4 | 5 | 6 | 2 | 3 | 4 | 5 | 6 |
| Average | 70 | 81 | 51 | 0 | 149 | 49 | 124 | 66 | 54 | 114 |
| Pressure kPa | | | | | | | | | | |
| Maximum | 185 | 140 | 80 | 0 | 265 | 150 | 205 | 135 | 190 | 195 |
| Pressure kPa | | | | | | | | | | |
| Minimum | 10 | 10 | 15 | 0 | 10 | 10 | 10 | 10 | 10 | 10 |
| Pressure kPa | | | | | | | | | | |
| Median | 60 | 85 | 55 | 0 | 155 | 35 | 135 | 60 | 40 | 115 |
| Mode | 80 | 70 | 60 | 0 | 240 | 10 | 125 | 55 | 30 | 70 |
| SD | 48 | 33 | 16 | 0 | 78 | 39 | 56 | 26 | 37 | 55 |

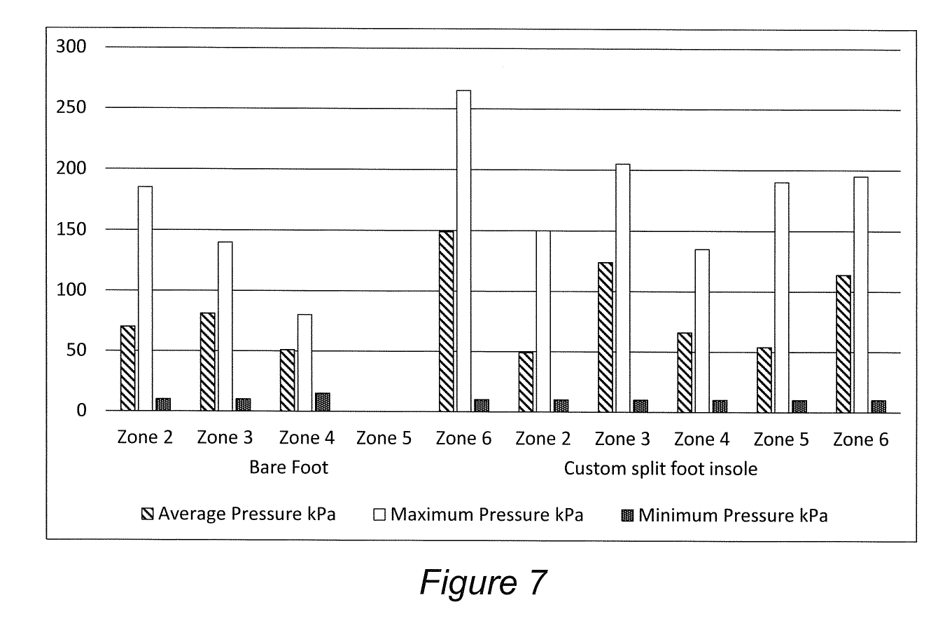

A graphical representation of the comparison of FIG. 5 showing barefoot pressure with FIG. 6 showing the pressure distributed using the split foot insole is as shown in FIG. 7.

The pressure build up in the various areas of each zone of plantar surface is as in FIG. 5 and the pressure offload after using the inventive custom made split insole as in FIG. 6, indicated in various colours highlighting the pressure rating as depicted in Table 2 here under.

TABLE 2
Analysis of FIG. 5

| S. No | Colour | Pressure Build up | Pressure Rating |
|---|---|---|---|
| 1 | Pink | 300 kPa and above | Dangerously high |
| 2 | Red | 220 kPa to 299 kPa | Very high |
| 3 | Yellow | 150 kPa to 219 kPa | High |
| 4 | Green | 100 kPa to 149 kPa | Normal |
| 5 | Light blue | 60 kPa to 99 kPa | Moderate |
| 6 | Dark blue | 30 kPa to 59 kPa | Low |
| 7 | Black | 10 kPa to 29 kPa | Very low |
| 8 | White | | No |

From the above Table 2 which is an analysis of FIG. 5 it can be seen that the areas highlighted in pink, red and yellow are more vulnerable to nerve and tissue damage and the chances of ulcer formation is very high. More is the area of pink colour, the chances of ulcer formation is highest and once formed ulcer would be very severe and chances of healing is very poor, which directly increases the chance of gangrene formation and amputation.

Therefore, pink and red colour is directly proportional to high risk ulcer formation, and poor healing and thereby leading to high possibility of gangrene formation. So the hexagonal and oval projections offload the pressure build up in the pink, red and yellow areas to the other normal, moderate or low pressure areas so as to offload the pressure build up in the said areas.

When a person is static wearing the custom made split insole, the peak pressure applied by his foot on the split insole would first be sensed in the fifth zone, then by the sixth zone, then by the third, second and fourth respectively, and then distributed to the entire foot, as shown in FIG. 6. The peak pressure is in zone 6 FIG. 5. The distributed peak pressure is in zone 3 shown in FIG. 6.

The application of peak pressure in the bare foot and its distribution in the split foot insole, is as shown in FIGS. 5 and 6 respectively. The numerically illustration of the same is as in Table 3 below;

TABLE 3
Zone wise pressure distribution

| Pressure Values | Bare Foot | Customized Insole |
|---|---|---|
| kPa | Pressure Zone | Pressure Zone |
| 300 and above | — | — |
| 220-299 | Zone 6 | — |
| 150-219 | Zone 2 & 6 | Zone 2, 3, 5 & 6 |
| 100-149 | Zone 2, 3, & 6 | Zone 2, 3, 5 & 6 |
| 60-99 | Zone 2, 3, 4 & 6 | Zone 2, 3, 4, 5 & 6 |
| 30-59 | Zone 2, 3, 4 & 6 | Zone 2, 4 & 5 |
| 10-29 | Zone 2, 3, 4 & 6 | Zone 2, 4 & 5 |
| Contact Area sq.cm | 135 | 158.5 |

The above Table 3 describes the pressure values in kPa, which is followed by zone wise pressure point distribution for both bare foot and during custom split insole application. From the above, it is evident that the custom split insole increases the surface area of contact between the foot and the insole, thereby leading to offloading of the peak pressure from few particular points in the feet to the entire feet, thereby reducing stress concentration to the soft foot tissue and the nerve at those particular points, which is the major cause for foot ulceration in diabetic patients, whose, foot is already affected by peripheral neuropathy and tissue damage, due to concentration of peak pressure at a few particular points in the foot.

### Advantages of the Present Invention

1. Replace-ability of the individual splits which are worn out is possible, rather than replacing the entire insole. This reduces cost of replacement and longer usage of the un-worn out splits.
2. Comfortability of use as the entire custom split insole is custom made, to the size of the user.
3. The deformation potential of VeroClear RGD810, TangoPlus FLX930 resin from which the split insoles are made is much less than the other insoles that are available for sports and other orthotic applications, so the life of these split insoles made from this resin is much more, when compared to that of others.
4. Inventory holding cost of raw material is drastically reduced,
5. Wastage of raw material is negligible in the present invention whereas it is quite high in conventional manufacturing processes.
6. Life of insole is high as only worn out parts need to be replaced and not the entire insole.
7. Effective distribution of plantar pressure is achieved.

We claim:

1. A custom made split foot insole, consisting of 6 zones for normal and flat foot persons and 5 zones for high arch foot persons, where the splits have projections, hexagonal or oval, of specific dimension and specific spacing but of uniform height; elasticity which varies from zone to zone.

2. An invention as in claim 1, where the zones 2 to 6 for normal and flat foot persons is placed on the first zone, which forms the bottom part of the split insole.

3. An invention as in claim 1, where the zones 2 to 5 for high arch foot persons, is placed on the first zone, which forms the bottom part of the split insole.

4. An invention as in claim 1, where the zones of split insole 2, 3, 4 and 6 for normal and flat foot persons are of uniform thickness of 4 mm and the first split insole is of uniform thickness 2 mm.

5. An invention as in claim 1, where the zones of split insole 2, 3 and 5 for high arch foot persons are of uniform thickness of 4 mm and the first split insole is of uniform thickness 2 mm.

6. An invention as in claim 1, where the projections in each split are of uniform height of 1 mm.

7. An invention as in claim 1, where the projections in zone 2 are of hexagonal pattern with distance between the hexagons perforation spacing 3 mm and angle 60° and their diameter in this zone is 2.5 mm.

8. An invention as in claim 1, where the spacing of the projections in zone 3 is located only in the middle of this zone. Around the projections there are horizontally placed oval projections of varying dimensions. This size and placement of these oval projections depends upon the peak pressure acting on the person's foot.

9. An invention as in claim 1, where the projections in split zone two are so made so that they come under the toes of the foot.

10. An invention as in claim 1, where the elasticity of the split insole in each zone, is directly proportional to the peak pressure applied at that point.

11. An invention as in claim 1, where there are two mounts of size (7 mm height, 8 mm length and 8 mm width) made on the surface of zone one, in such a manner that it can be inserted in the square socket in zone five, which is of appropriate size to take these mounts.
